SEQUENCE LISTING (3 sequences)

SEQ ID NO: 1 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 2 - protein, 216 aa, Homo sapiens
MLRLSERNMKVLLAAALIAGSVFFLLLPGPSAADEKKKGPKVTVKVYFDLRIGDEDVGRVIFGLFGKTVPKTVDNFVALATGEKGFGYKNSKFHRVIKDFMIQGGDFTRGDGTGGKSIYGERFPDENFKLKHYGPGWVSMANAGKDTNGSQFFITTVKTAWLDGKHVVFGKVLEGMEVVRKVESTKTDSRDKPLKDVIIADCGKIEVEKPFAIAKE

SEQ ID NO: 3 - protein, 212 aa, Homo sapiens
MGPGPRLLLPLVLCVGLGALVFSSGAEGFRKRGPSVTAKVFFDVRIGDKDVGRIVIGLFGKVVPKTVENFVALATGEKGYGYKGSKFHRVIKDFMIQGGDITTGDGTGGVSIYGETFPDENFKLKHYGIGWVSMANAGPDTNGSQFFITLTKPTWLDGKHVVFGKVIDGMTVVHSIELQATDGHDRPLTNCSIINSGKIDVKTPFVVEIADW

(19) United States
(12) Patent Application Publication
(10) Pub. No.: US 2009/0258024 A1
(43) Pub. Date: Oct. 15, 2009

(54) COMPOSITIONS AND METHODS FOR DIAGNOSIS AND TREATMENT OF CHRONIC INFLAMMATORY DISEASES

(75) Inventors: MICHAEL BUKRINSKY, POTOMAC, MD (US); STEPHANIE CONSTANT, HERNDON, VA (US)

Correspondence Address: LAW OFFICES OF KHALILIAN SIRA, LLC, 9100 PERSIMMON TREE ROAD, POTOMAC, MD, 20854, US

(73) Assignee: THE GEORGE WASHINGTON UNIVERSITY, WASHINGTON, DC (US)

(21) Appl. No.: 12/405,948

(22) Filed: Mar. 17, 2009

Related U.S. Application Data
(60) Provisional application No. 61/037,192, filed on Mar. 17, 2008.

(51) Int. Cl. A61K 39/395; A61K 38/16; A61K 38/13; A61P 29/00

(52) U.S. Cl. 424/141.1; 514/12; 514/11

(57) ABSTRACT

This invention relates to methods and compositions for diagnosis and treatment of chronic inflammatory diseases by blocking CD147 interaction with extracellular cyclophilin. Specifically, the methods and compositions of this invention regulate recruitment of leukocyte to the infection site by specifically blocking the CD147 domain involved with the chemotactic function without blocking the CD147 domain involved with EMMPRIN function.

### CROSS REFERENCE TO RELATED APPLICATIONS

This application claims priority to provisional application No. 61/037,192, filed Mar. 17, 2008, the content of which is incorporated herein by its entirety

### STATEMENT REGARDING FEDERALLY SPONSORED RESEARCH OR DEVELOPMENT

Federal government funds were used in researching or developing this invention, namely NIH grant R21-AI60720, and AHA predoctoral fellowship 0615392U (JMD). The George Washington University and The National Institute of Health are parties to the joint Research Agreement

### FIELD OF THE INVENTION

This invention relates methods and compositions for amelioration, treatment and diagnosis of inflammatory diseases. In particular, the invention is directed to methods and compositions that interfere with CD147 interaction with extracellular cyclophilin without blocking the CD147 domain involved with extracellular matrix metalloproteinase inducer function.

### I. BACKGROUND OF THE INVENTION

The recruitment and trafficking of leukocytes is an essential aspect of the inflammatory process. Although chemokines are thought to be the main regulators of cell trafficking, extracellular cyclophilins have recently been shown to have potent chemoattracting properties for human leukocytes. Cyclophilins are secreted by a variety of cell types and are detected at high levels in tissues with ongoing inflammation. CD147 has been identified as the main signaling receptor for cyclophilin A on human leukocytes.

Cyclophilins are ubiquitously distributed intracellular proteins first recognized as the host cell receptors for the potent immunosuppressive drug, Cyclosporine A. While most studies have previously focused on the intracellular activities of cyclophilins, accumulating evidence suggests a role for these proteins as mediators of intercellular communication [1, 2]. It has been demonstrated that secreted cyclophilin A (CypA) is a potent leukocyte chemoattractant in vitro [3-5]. CypA has also been shown to elicit inflammatory responses, characterized by a rapid influx of leukocytes, when injected in vivo [4].

CD147 has previously been identified as a type I transmembrane protein and the receptor for extracellular cyclophilins. It has also been shown that CD147 is involved in cyclophilin-mediated events. [3]. However, the relevance of these findings to physiological or pathological conditions of inflammatory diseases has not been previously disclosed.

It has recently been reported that treatment with anti-CD147 mAb could reduce leukocyte influx to inflamed lungs in mouse models of acute lung injury [13] and allergic asthma [14]. In vitro studies looking at the capacity of extracellular cyclophilins to promote integrin-mediated adhesion of T lymphocytes to extracellular matrix proteins have demonstrated differences in responsiveness to cyclophilins, correlating with the differentiation status of the T cells [15].

These findings, however, do not address the issue of whether extracellular cyclophilins have the capacity to interact more readily with activated populations of lymphocytes and whether such activity might be CD147-mediated. Furthermore, the literature does not provide any solutions as how to interfere with the immune system and to avoid the risk of secondary infections resulting from compromised immune functions. There is a long felt need in the industry to address these and other medical issues when dealing with inflammatory diseases through regulation of T cells and their cell receptors.

The invention disclosed herein addresses these and other needs by disclosing and examining the capacity of cyclophilin A to induce the migration of human CD4⁺T cells and establish the pathway of responses that relate to CD147 expression. The invention disclosed herein provides for compositions and methods that treat and/or ameliorate symptoms of inflammatory diseases by blocking CD147 interaction with extracellular cyclophilin, and specifically blocking the CD147 domain involved with the chemotactic function without blocking the CD147 domain involved with EMMPRIN function.

### II. SUMMARY OF THE INVENTION

This invention relates to compositions and methods for diagnosis, treatment or amelioration of one or more symptoms of a chronic inflammatory disease.

In one aspect, the invention provides a method of treating symptoms of chronic inflammatory disease in a subject in need thereof by administering a therapeutically effective amount of a compound that blocks CD147 interaction with an extracellular cyclophilin without blocking the CD147 domain involved with EMMPRIN function, wherein the compound regulates recruitment of leukocytes to a target tissue and thereby treats or ameliorates symptoms of the chronic inflammatory disease. The compounds of the invention regulate recruitment of leukocytes to a target tissue. The target tissue comprises the tissues at the site of inflammation or tissues that are otherwise implicated directly or indirectly in the inflammation process.

In one embodiment, the compound is a derivative of CD147, a derivative of cyclophilin, or a combination thereof.

In another embodiment, the derivative of CD147 comprises an antagonist of CD147.

In yet another embodiment, the antagonist of CD147 comprises a monoclonal antibody against CD147.

In one embodiment, cyclophilin comprises cyclophilin A a derivative of cyclosporine A (CsA).

The chronic inflammatory diseases within the scope of the invention include a wide variety of inflammatory diseases such as, by way or example and not limitation, Osteoarthritis, Chronic Obstructive Pulmonary Disease, chronic inflammatory connective tissue diseases, lupus, scleroderma, Sjogrens' syndrome, poly- and dermatomyositis, vasculitis, chronic inflammatory bowel disease, multiple sclerosis, rosacea, chronic pelvic inflammatory disease, Crohn's disease, chronic inflammatory polyneuropathy, Rheumatoid arthritis, and neovascular diseases of the eye, among others.

In yet another embodiment, the compound is a non-immunosuppressive cyclosporine A (CsA) derivative modified to inhibit interaction with extracellular cyclophilin.

According to another aspect, the invention discloses drug screening methods for selecting compounds that inhibit binding between CD147 and external cyclophilins without blocking the CD147 domain involved with EMMPRIN function. The drug screening methods comprise contacting the CD147 or cyclophilin polypeptide or a variant thereof with a library of compounds suspected of having antagonist or agonist activity with CD147 and/or external cyclophilins, identifying select compounds that bind to or otherwise interact with CD147 and/or cyclophilins, and assaying the biological activity of the select compounds, wherein the selected compounds reduce or inhibit recruitment of leukocytes to a target tissue.

According to yet another aspect, the invention provides pharmaceutical compositions for treatment of chronic inflammatory diseases comprising a therapeutically effective amount of a compound that blocks or otherwise interacts with binding between CD147 and an extracellular cyclophilin without blocking the CD147 domain involved with EMMPRIN function, and a suitable carrier or diluent.

According to yet another aspect, the invention provides diagnostic test kits to diagnose, detect and/or quantify an inflammatory disease in a subject in need thereof, comprising a compound that blocks binding between CD147 and an extracellular cyclophilin without blocking the CD147 domain involved with EMMPRIN function, inactive agents used for biological assays including buffers, minerals, and water, among others, instructions for the use of the compound and the biological assays to predict or diagnose the occurrence or recurrence of an inflammatory mediated disease in the subject.

These and other aspects, features and advantages of the present invention will become apparent after a review of the following detailed description of the disclosed embodiments and the appended claims.

### III. BRIEF DESCRIPTION OF THE DRAWINGS

FIG. 1 is a three part histogram (A), (B), and (C) and shows CD147 expression and cyclophilin A (CypA) in Collagen-induced arthritis (CIA). FIG. 1(A) shows that neutrophils, monocytes and activated CD4+ T cells were stained with anti-CD147, or isotype control antibody, followed by FITC-conjugated anti-rat IgG. Neutrophils and monocytes were distinguished based on forward scatter/side scatter characteristics and CD4+ T cells were identified by co-staining with Cy5-anti-CD4. Histograms show CD147 staining (open) versus isotype staining (filled). FIG. 1(B) is a line graph and shows the results when DBA/1J mice were immunized with collagen in CFA and then challenged three weeks later with collagen in PBS. Starting 7 days after challenge, inflammation was scored for individual joints. Data show mean (+SE) clinical scores with n=6 mice per time point. FIG. 1(C) is a bar graph and shows that groups of mice were sacrificed when they reached specific CIA scores and proteins extracted from joints were analyzed by Western blot analysis using anti-CypA Ab. Data show mean band densitometry of lysates from naïve mice vs. mice with intermediate or high CIA clinical scores.

FIG. 2 is a two part set of bar graphs. FIG. 2A has three panels, FIG. 2B has two panels. FIG. 2 shows that leukocyte migration mediated by CypA is blocked by RL73.2 anti-CD147. FIG. 2(A) shows that purified neutrophils, monocytes and CD4+ T cells were set up in Boyden chemotaxis chambers in the presence of extracellular CypA with/without anti-CD147 or isotype control mAb. Bar graphs show mean (+SE) chemotactic index for each group, with n=4-6 wells per group. Horizontal dashed lines denote the cutoff chemotactic index for significant migration, set at 1.25. FIG. 2(B) shows that MC57 mouse fibroblasts and purified mouse macrophages were co-cultured in the presence of RL73.2 anti-CD147, or UM-8D6 anti-CD147, or relevant isotype control mAbs. After 72 hours, culture supernatants were tested for pro-MMP-9 (pro-Matrix Metalloproteinase-9) by ELISA. Bar graphs show mean (+SE) concentrations of pro-MMP-9 detected in cultures. Statistical differences between anti-CD147 and isotype groups were established using a Student's t-test: ***=p<0.001 and **=p<0.01.

FIG. 3 has three panels that include 2 line graphs and 1 set of bar graphs, and shows anti-CD147 intervention reduces the severity of CIA. DBA1/J mice were immunized and challenged with collagen II to induce CIA. FIG. 3 (A) Half the mice were treated every 3 days starting on day 21 with RL73.2 anti-CD147 mAb while the other half were treated with isotype control mAb. Graph shows the mean (+SE) score for each group (n=6 per group). FIG. 3(B) shows that half the mice were treated on days 21-30 with RL73.2 anti-CD147 mAb while the other half were treated with an isotype control mAb. Graph shows the mean (+SE) score for each group (n=9 per group). FIG. 3(C) shows that mice were sacrificed at the peak differential between anti-CD147 and isotype treatment and joint proteins were extracted. Myeloperoxidase (MPO) levels were established using TMB substrate and TNFα levels were measured by ELISA. A Student's t-test was used to establish statistical significance at individual time points or between groups: **=p<0.01; *=p<0.05.

FIG. 4 is a two panel set of graphs relating to data using a non-immunosuppressive cyclosporine A derivative (NI-CsA) in a mouse model of acute allergic asthma.

FIG. 5 is a line graph showing intervention using NI-CsAin a mouse model of CIA—early treatment.

FIG. 6 is a line graph showing intervention using NI-CsAin a mouse model of CIA—later treatment.

### IV. DETAILED DESCRIPTION OF THE INVENTION

The invention as claimed herein demonstrates that CD147-cyclophilin interactions contribute to the pathogenesis of inflammatory disease. It has been found unexpectedly that activated human T lymphocytes express elevated levels of CD147, compared to resting T cells, and that these activated T cells migrate more readily to cyclophilin A than resting cells. The invention also discloses that unlike resting CD4+ T cells, the cyclophilin-mediated migration of activated T cells does not require interaction with heparin sulphate receptors, but instead is dependent on CD147 interaction alone. These unexpected findings indicated that cyclophilin-CD147 interactions will be most potent when leukocytes are in an activated state, for example during inflammatory responses. Thus, targeting cyclophilin-CD147 interactions will provide a novel approach for alleviating tissue inflammation.

Without being limited to any particular mechanism of action, one mechanism for CD147-cyclophilin interaction and pathogenesis of an inflammatory disease is by promoting the recruitment of leukocytes into the inflammatory tissue or target site. Importantly, the CD147-cyclophilin interaction according to this invention can be specified according to the domain that is interacted with such that side effects can be reduced.

As used herein a “derivative of CD147” or a “derivative of the “extracellular cyclophilin” includes analogs, allelic variants, substituted and/or mutated molecules including peptide-based molecules, nucleic acid-based molecules and/or small molecules. The peptide-based and nucleic acid based molecules are any nucleic acid molecule, protein, polypeptide or peptide fragment that is produced in the course of the transcription, reverse-transcription, polymerization, translation, post-translation and/or expression of a nucleotide molecule encoding CD147 or a cyclophilin. The derivatives also include, for example, biologically active fragments, substantially homologous polypeptides, oligopeptide, homodimers, heterodimers, substituted peptides, mutated peptides, variants of the polypeptides, modified polypeptides, analogs, fusion proteins, agonists, antagonists, antibody of the polypeptide, whether natural peptides, recombinant peptides, synthetic peptides, or a combination thereof. A derivative of CD147” or a “derivative of the “extracellular cyclophilin” also include one, two, three or more epitopes or fragments, whether contiguous or non-contiguous or non-contagious fragments, of the peptide represented by SEQ ID No: 1 or a nucleic acid molecule or fragments thereof encoding one or more fragments or epitopes represented by SEQ ID NO: 1

As used herein, “a fragment of peptide” includes fragments of about 10-200 amino acids or more. In one embodiment, the fragment preferably consists of amino acid sequences of no more than 200 amino acids. In some embodiments, the fragment consists of amino acid sequences of 10-100 amino acids. In a preferred embodiment, the amino acid sequences are in the range of 20-80 in length. In other specific embodiments, the fragments are in the range of 10-30 amino acids in length, in the range of 10-40 amino acids in length, in the range of 20-50 amino acids in length, in the range of 40-80 amino acids in length, in the range of 50-150 amino acids in length, in the range of 80-120 amino acids in length, among others.

It is intended herein that by recitation of such specified sizes, the sizes recited also include all those specific integer amounts between the recited sizes. For example, the size of 50-150 amino acid also encompasses 57-88, 67-98, etc, without actually reciting each specific range therewith.

As used herein, “non-immunosuppressive cyclosporine A (NI-CsA) derivatives” refers to CsA derivatives that are modified so as to inhibit or reduce the function of extracellular cyclophilins without affecting intracellular cyclophilin function.

As used herein, “regulation of leukocyte recruitment” refers to upregulation, or down regulation of leukocyte function or changes in the pattern of trafficking, influx, and/or migration of leukocytes to the site of inflammation. In one preferred embodiment of the invention, “regulation of leukocyte recruitment” refers to reduction in the migration of leukocytes to the site of inflammation.

As used herein, “target tissue” refers to the tissues or cells that are affected in a particular chronic inflammatory disease. Accordingly, target tissues within the scope of this invention include a wide variety of tissues that are at the site of inflammation or are otherwise implicated in an inflammatory disease.

As used herein, “treatment” refers to amelioration and or treatment of one or more symptoms of an inflammatory disease or a chronic inflammatory disease. Treatment may not be absolute so long as one symptom of the disease is ameliorated or treated.

As used herein “biologically active fragments” refer to fragments exhibiting activity similar, but not necessarily identical, to an activity of the CD147 or cyclophilin of the present invention. The biologically active fragments may have improved desired activity, or a decreased undesirable activity.

As used herein “nucleotide-based CD147 or cyclophilin derivatives” include cDNA, RNA, DNA/RNA hybrid, siRNA, anti-sense RNA, mRNA, ribozyme, and genomic DNA, among others.

As used herein “small molecules” include, but are not limited to, carbohydrates, carbohydratemimetics, peptidomimetics, organic or inorganic compounds (i.e, including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein “pharmaceutical composition” refers to a composition that contains one or more compounds that block binding between CD147 and cyclophilin and a pharmaceutically acceptable carrier or diluent. The “pharmaceutical composition” also refers to a composition that additionally contains a secondary drug or medicament. The “pharmaceutical composition” also refers to a composition that contains a variant of the compound including agonist, antagonist, biologically active fragments, analogs, modified peptides, substantially homologous sequences thereof.

As used herein “fusion protein” refers to a protein encoded by two or more, often unrelated, fused genes or fragments thereof. Membrane bound proteins, such as protein disulfide isomerase (PSI) are particularly useful in the formation of fusion proteins. Such proteins are generally characterized as possessing three distinct structural regions, an extracellular domain, a transmembrane domain, and a cytoplasmic domain. This invention contemplates the use of one or more of these regions as components of a fusion protein.

As used herein “inflammatory disease” refers to any human or animal disease or disorder, affecting any one or any combination of organs, cavities, or body parts, which is characterized by single or multiple local abnormal proliferations or inflammation of cells, groups of cells, or tissues, whether benign or malignant.

The invention described herein provides methods and compositions for diagnosis, and treatment of one or more symptoms of an inflammatory or chronic inflammatory disease in a variety of different infections. Because CD147 is expressed on all cell types, including hematopoietic, epithelial, endothelial, and tumor cells, the compositions and methods of this invention are useful to address a wider variety of inflammatory diseases affecting different cells and tissues.

The inflammatory diseases and chronic inflammatory diseases within the scope of the invention include, by way of example and not limitation, Osteoarthritis, Chronic Obstructive Pulmonary Disease, or COPD, chronic inflammatory connective tissue diseases (e.g., lupus, scleroderma, Sjogrens' syndrome, poly- and dermatomyositis, vasculitis), chronic inflammatory bowel disease, multiple sclerosis, rosacea, chronic pelvic inflammatory disease, Crohn's disease, chronic inflammatory polyneuropathy, Rheumatic diseases, neovascular diseases of the eye, including for example, neovascular glaucoma, diabetic retinopathy, retinoblastoma, retrolental fibroplasia, uveitis, retinopathy of prematurity macular degeneration, corneal graft neovascularization, as well as other eye inflammatory diseases, ocular tumors and diseases associated with choroidal or iris neovascularization, among others.

Rheumatoid arthritis is an inflammatory disease characterized by the infiltration of pro-inflammatory leukocytes such as neutrophils, monocytes, and activated CD4+ T cells into the joint space and tissue. These invading leukocytes secrete many cytokines, including TNFα and IL-1β, which activate resident fibroblast-like synoviocytes, resulting in fibroblast hyperproliferation and the production of tissue-degrading matrix metalloproteinases (MMPs)

The present invention as described herein demonstrates that CD147 functions as a regulator of leukocyte migration through its cell surface interaction with chemotactic extracellular cyclophilins. The role of CD147-cyclophilin interactions during inflammatory diseases, such as rheumatoid arthritis (RA), is described herein. CD147 is the principal cell-surface signaling receptor for the chemotactic activity of extracellular cyclophilins. Moreover, monoclonal antibodies specific for CD147 inhibited the in vitro migration of leukocytes mediated by extracellular cyclophilins, demonstrating the dependence on CD147 interaction for this chemotactic activity. Due to the relevance of CD147 and extracellular cyclophilins to leukocyte migration, we have found unexpectedly that CD147-cyclophilin interactions play a significant role in the development of inflammation, by promoting leukocyte infiltration into tissues during ongoing inflammatory responses.

In one embodiment of the invention, an anti-CD147 antibody induced a significant reduction or complete loss of the ability to migrate of pro-inflammatory leukocytes, specifically neutrophils, monocytes and activated CD4+ T cells in response to cyclophilin A in vitro. We have also shown that, in vivo treatment with CD147 mAb reduces the development of CIA significantly by about 75-90% in mice.

Chemokines are known to be critical factors for the recruitment and infiltration of inflammatory leukocytes from the circulation into joint tissues. Indeed, many different chemokines including IL-8, monocyte chemotactic protein-1 (MCP-1), macrophage inflammatory protein 1α (MIP-1α), and regulated upon activation normal T cell expressed and secreted (RANTES), have been detected in the synovial fluid of RA patients.

In one embodiment, the capacity of CD147 to stimulate MMP secretion has been examined in the context of different inflammatory diseases. For example, in vitro studies have demonstrated that cell-surface CD147 on pro-inflammatory leukocytes, such as macrophages, can interact with CD147 expressed on synovial fibroblasts and induce the production of MMPs that contribute to RA pathology. Other than its EMMPRIN activity, CD147 exhibits additional functions that may also contribute to RA. One of these is a capacity to interact with extracellular proteins, most notably extracellular cyclophilins.

Cyclophilins are a family of ubiquitously expressed intracellular proteins functioning as peptidyl-prolyl cis-trans isomerases. Cyclophilin A (CypA) is the best characterized and most abundant of the cyclophilins, accounting for 0.1-0.4% of total cellular protein. Additionally, CypA has been identified as the intracellular binding partner for the immunosuppressive drug, cyclosporine A. Importantly, cyclophilins can be actively secreted, making these proteins able to function in an extracellular manner. Extracellular cyclophilins have been shown to possess potent chemotactic capacity for several human and mouse leukocyte subsets, including neutrophils, T-cells, and monocytes. Thus, cyclophilins represent a novel family of extracellular proteins with the ability to function as chemokines.

In one embodiment, the invention encompasses derivatives of cyclosporine A (CsA) which are altered to prevent their interaction with intracellular cyclophilins. CsA may be derivetized by methods including, for example, methods involving the addition of a bulky substituent to the protein. Examples of such substituents, include but are not limited to, charged substituents (e.g., spermine or spermidine), polynucleotides with and without modified backbones, carbohydrates (e.g., polyacrylic acid, polysodium acrylate, polycesium acrylate, polymethacrylic acid), amphiphilic block copolymers (e.g., polystyrene poly) (sodium acrylate), and amphiphilic homopolymers.

Non-immunosuppressive cyclosporine A (NI-CsA) analogs or derivatives contemplated within the scope of the invention may be made according to well known principles of peptide modification and/or replacement, with the proviso that they should be functionally capable of interfering with extracellular CD147-cyclophilin interactions.

In one preferred embodiment of the invention, cyclosporine A (CsA) is derivatized by reaction with polyethylene glycol, resulting in a “pegylated” CsA. Pegylated CsA may be prepared by standard chemical methods known to those skilled in the art. One method of preparing the pegylated CsA of the invention involves reacting methoxypolyethylene glycol-succinimidyl succinate with 8-amino-cyclosporin A and 4-dimethylamino pyridine in methylene chloride with stirring for two days at room temperature in the dark. To block any unreacted sites, ethanolamine was then added and the mixture incubated at room temperature with stirring for another 24 hours. The derivatized CsA is purified from the reaction mixture by normal phase HPLC.

The invention as disclosed herein demonstrates that the level of extracellular CypA within the synovial fluid of RA patients were found to directly correlate with the number of neutrophils present in the same fluid and CD147-cyclophilin interactions contributed to chronic types of inflammatory responses.

In one embodiment, the collagen-induced arthritis (CIA) mouse model has been used that shares many of the clinical and pathological features of human RA, including an infiltration of inflammatory leukocytes into the synovium. The data demonstrated that treating CIA mice with anti-CD147 either at the onset, or throughout CIA disease reduces joint inflammation by 75-90%. Further findings herein demonstrate that a non-immunosuppressive CsA analog was also effective at inhibiting CD147-cyclophilin interaction. These findings provide a potentially novel target (CD147-cyclophilin interactions) to consider for reducing tissue inflammation in RA, as well as other types of chronic inflammatory diseases.

Drug Screening

Further contemplated within the scope of the present invention is the use of drug screening methods to screen for compounds that blocks CD147 and extracellular cyclophilin interaction without blocking the CD147 domain involved with EMMPRIN. This invention is particularly useful for screening therapeutic compounds by using a CD147 and or cyclophilin protein or a derivative thereof, or a binding fragment thereof, in any of a variety of drug screening techniques. In one embodiment, the screening method is used for identifying polypeptides, nucleotide and/or small molecules that blocks CD147-cyclophilin interaction. Such a drug screening method would include, for example, contacting the CD147 or cyclophilin polypeptide or a variant thereof with a selected compound(s) suspected of having antagonist or agonist activity, and assaying the activity of these compounds following binding. These binding molecules are useful, for example, as agonists and antagonists and can be used, in accordance with the invention, in the therapeutic embodiments described in detail, below.

The compounds employed in such a test may be affixed to a solid support, expressed on a cell surface, free in solution, or located intracellularly. One method of drug screening utilizes eukaryotic or prokaryotic host cells which are stably transformed with recombinant nucleic acids expressing the polypeptide or fragments thereof. Drugs are screened against such transformed cells in competitive binding assays. One may measure, for example, the formulation of complexes between the agent being tested and a polypeptide or variant of the present invention.

Thus, the present invention provides methods of screening for drugs or any other agents which affect activities mediated by the CD147-cyclophilin interaction of the present invention. These methods comprise contacting such an agent with a compound of the present invention and assaying for the presence of a complex between the agent and the compound, by methods well known in the art. In such a competitive binding assay, following incubation, free agent is separated from that present in bound form, and the amount of free label is a measure of the ability of a particular agent to inhibit binding between CD147 and cyclophilin according to the present invention.

Another technique for drug screening provides high throughput biological or chemical libraries for screening for compounds having suitable binding affinity to the compounds of the present invention, and is described briefly herein. Large numbers of different small peptide test compounds are synthesized on a solid substrate, such as plastic pins or some other surfaces. The peptide test compounds are reacted with a known compound of the present invention and washed. Bound polypeptides are then detected by methods well known in the art. Purified polypeptides are coated directly onto plates for use in the aforementioned drug screening techniques. In addition, non-neutralizing antibodies may be used to capture the peptide and immobilize it on the solid support.

This invention also contemplates the use of competitive drug screening assays in which neutralizing antibodies are used that are capable of binding compounds of the present invention and specifically compete with a test compound for binding to CD147 and/or cyclophilins. In this manner, the antibodies are used to detect the presence of any peptide which shares one or more antigenic epitopes with a compound of the invention.

In certain situations, it may be desirable to wash away any unbound polypeptide of the invention, or alternatively, unbound polypeptides, from a mixture of the polypeptide of the invention and the plurality of polypeptides prior to attempting to determine or to detect the presence of a selective affinity interaction. Such a wash step may be particularly desirable when the polypeptide of the invention or the plurality of polypeptides is bound to a solid support.

In one embodiment, the drug screening methods are provided by way of diversity libraries, such as random or combinatorial peptide or non-peptide libraries which can be screened for molecules that specifically blocks CD147 and cyclophilin interaction. Many libraries are known in the art that can be used, i.e., chemically synthesized libraries, recombinant (i.e., phage display libraries), and in vitro translation-based libraries. Examples of chemically synthesized libraries are described in Fodor et al., Science 251:767-773 (1991); Houghten et al., Nature 354:84-86 (1991); and Brenner and Lemer, Proc. Natl. Acad. Sci. USA 89:5381-5383 (1992), among others.

Diagnostic Methods and Test Kits

The antibodies and derivatives of CD147 and/or cyclophilins peptides of the present invention are also used in diagnostic methods and kits to diagnose, detect or quantify an inflammatory disease in patients or their biological sample or specimen. Results from these tests can be used to predict or diagnose the occurrence or recurrence of an inflammatory mediated disease. Antibodies to the CD147 peptide may also be used in production facilities or laboratories to isolate additional quantities of CD147 derivatives, such as by affinity chromatography, or for the development of peptide agonists or antagonists.

Pharmaceutical Composition

The present invention also provides pharmaceutical compositions comprising a therapeutically effective amount of a compound of the invention and a pharmaceutically acceptable carrier.

According to one embodiment, the compounds of the invention having anti-inflammatory activity described herein are provided as isolated and substantially purified compounds in pharmaceutically acceptable formulations using formulation methods known to those of ordinary skill in the art. These formulations can be administered by standard routes.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The pharmaceutical composition formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

The amount of the compound of the invention which will be effective in the treatment or amelioration of one or more symptoms of an inflammatory disease be determined by standard clinical techniques. In addition, in vitro assays may optionally be employed to help identify optimal dosage ranges.

In particular, the dosage of the compounds of the present invention will depend on the disease state or condition being treated and other clinical factors such as weight and condition of the human or animal and the route of administration of the compound. The precise dose to be employed in the formulation, therefore, should be decided according to the judgment of the practitioner and each patient's circumstances. Effective doses may be extrapolated from dose-response curves derived from in vitro or animal model test systems.

For treating humans or animals, between approximately 0.5 to 500 mg/kilogram is typical broad range for administering the pharmaceutical composition of the invention. The methods of the present invention contemplate single as well as multiple administrations, given either simultaneously or over an extended period of time. It is to be understood that the present invention has application for both human and veterinary use.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Animals, Antibodies and Reagents

In vivo studies were conducted using male DBA/1J mice (age 9-10 weeks) purchased from Jackson Laboratories (Ben Harbor, Me.). In vitro studies were conducted using female C57BL/6 mice aged 6 weeks or older purchased from the National Cancer Institute (Bethesda, Md.). All studies were approved by the Institutional Animal Care and Use Committee at The George Washington University Medical Center.

Immunization grade bovine collagen II (CII) and Complete Freund's Adjuvant (CFA) were purchased from Chondrex (Redmond, Wash.). Rat anti-mouse CD147 monoclonal antibody was purified from the RL73.2 hybridoma originally donated to us by H. R. MacDonald (Ludwig Institute for Cancer Research, Switzerland). The rat IgG2a hybridoma (HB-189) obtained from the American Type Culture Collection (Manassas, Va.) was used as a source of isotype control antibody. Both mAbs were purified by the National Cell Culture Center (Minneapolis, Minn.). The UM-8D6 clone and its corresponding IgG1 isotype control were purchased from Ancell (Bayport, Minn.). MC57 fibroblasts were maintained in RPMI+L-glutamine+10% FBS. Fluorescein-conjugated F(ab′)2 anti-rat IgG (secondary) Ab was purchased from Jackson ImmunoResearch Laboratories (West Gove, Pa.). Cy-Chrome-conjugated anti-mouse CD4 mAb was purchased from BD Biosciences (Franklin Lakes, N.J.). Rabbit anti-cyclophilin A antibody was obtained from U.S. Biological (Swampscott, Mass.). Horseradish peroxidase (HRP)-conjugated anti-rabbit secondary Ab was purchased from Amersham Biosciences (Piscataway, N.J.). TMB substrate was purchased from Dako (Carpinteria, Calif.). Human recombinant CypA was purchased from Calbiochem (San Diego, Calif.). Bovine serum albumin (BSA) fraction V and concanavalin A (ConA) were purchased from Sigma (St. Louis, Mo.).

### Example 2

### Regimen for Induction of CIA

Male DBA/1J mice were immunized with 100 μg CII emulsified in CFA on day 0 via a tail-base injection. On day 21, immunized mice were boosted via an i.p. injection with 100 μg CII in PBS. Untreated mice were used in some experiments as a negative control. To evaluate severity of the disease, a macroscopic clinical scoring method was used. Scoring took place every 3-4 days with the following scale: 0=normal joint; 1=mild swelling and/or redness; 2=pronounced edema or redness of the paw or several digits; 3=severe swelling of entire paw. A clinical score was generated for each mouse by combining the score of all 4 paws (maximum score of 12).

### Example 3

### Anti-CD147 Intervention Regimens

For all intervention studies, doses of 5 μg of anti-CD147, or isotype control mAb, were administered per injection by i.p. delivery in 100 ul PBS. This dose was determined to be optimal based on preliminary titration studies. For the studies in which anti-CD147 intervention was conducted throughout disease, antibody was administered starting on the day of CII challenge and then every 3 days until sacrifice. For the studies in which anti-CD147 intervention was given at the onset of disease, antibody was given daily for 10 days starting on the day of CII challenge. Animals were sacrificed at various times during the course of disease for tissue analysis or once disease had resolved (see Figure legends).

### Example 4

### Analysis of Joint Proteins

At indicated time points during disease, animals were sacrificed and joint tissues were isolated, cleaned, and then frozen at −80° C. until analysis. Frozen joint tissues were pulverized on dry ice and protein was extracted using RIPA buffer (50 mmol/L Tris-HCL, pH 7.5, 150 mmol/L NaCl, 1% Nonidet P-40, 0.5% sodium deoxycholoate, 0.1% sodium dodecyl sulfate). For western blot analysis, equal volumes of protein samples were fractionated by 4-20% SDS-PAGE, blotted onto nitrocellulose membrane, and then probed with anti-CypA antibody. Densitometric analysis of developed autoradiograph bands was conducted using Molecular Dynamics Personal Densitometer SI. To measure levels of myeloperoxidase (MPO), 50 μl of joint protein extract was combined with 50 μl of TMB substrate and the resulting calorimetric reaction was quantified using an ELISA plate reader. TNF-α levels were measured by ELISA with a kit purchased from R&D Systems (Minneapolis, Minn.).

### Example 5

### Leukocyte Isolation and Generation

Neutrophils were isolated from the peritoneal cavity of mice 3 hours after i.p. injection of 1 ml of 3% thioglycolate broth. Isolated peritoneal cells were washed 3 times with cold PBS and then overlaid onto a sequential gradient of Histopaque-1007 and -1119 (Sigma, St. Louis, Mo.). Enriched neutrophils (>80%) were collected from the interface of the two Histopaque layers. Monocytes were isolated from blood collected via cardiac puncture. Peripheral blood mononuclear cells (PBMCs) were enriched by centrifugation over Lymphocyte Separating Medium (LSM; Mediatech Inc., Herndon, Va.) and the monocytes were then enriched to 90% by MACS separation using a positive selection kit specific for CD11b+cells (Miltenyi Biotec, Auburn, Calif.). Activated CD4+ T cells were generated by overnight stimulation of total splenocytes (3×106 cells/well) with ConA (1 μg/ml). The CD4+ T cells were then purified from these populations by MACS negative selection kit. Naïve CD4+ T cells were purified from populations of unstimulated spleen cells.

### Example 6

### Chemotaxis Assays

Chemotaxis assays were conducted using 48-well modified Boyden chambers (Neuroprobe, Gaithersburg, Md.) with the two compartments separated by a 5-μm polycarbonate membrane (Neuroprobe). The chemotaxis of purified neutrophils, monocytes and CD4+ T cells was assessed by adding 104 cells in medium (RPMI 1640+1% BSA) to the upper compartment and medium containing 100 ng/ml CypA for neutrophils, 200 ng/ml CypA for monocytes, and 400 ng/ml CypA for CD4+ T cells to the wells of the lower compartment. (These doses were established to be optimal for each cell subset by preliminary titration studies). For blocking experiments, 25 μg/ml anti-CD147 or isotype control was added to each compartment for the neutrophil experiments and 10 ug/ml anti-CD147 or isotype for the monocyte and CD4+ T cell experiments. Lower compartment wells containing medium alone were utilized as a negative control. The loaded chemotaxis chambers were incubated at 37° C. in 5% CO2 for 50 min. Following incubation, the membrane was removed, non-migrated cells were scraped off, and the membrane was stained with Wright-Giemsa (CAMCO, Fort Lauderdale, Fla.). A chemotactic index was generated for each well by dividing the number of cells migrating within each test well by the average number of cells migrating to medium alone.

### Example 7

### Analysis of CD147 Expression on Leukocytes

The expression of CD147 on neutrophils and monocytes was analyzed on peripheral blood leukocytes (PBLs) obtained from blood collected via cardiac puncture. Red blood cells were removed by treating blood with RBC lysis buffer (1.6 g ammonium chloride, 0.2 g potassium bicarbonate, and 0.03 g EDTA in 100 ml distilled H20). Activated CD4+ T-cells were generated as described above. All cell populations were stained with anti-mouse CD147, or isotype control mAb, followed by FITC-conjugated anti-rat IgG (secondary). Flow cytometric analysis was performed using forward scatter/side scatter plots to distinguish individual populations.

### Example 8

### MMP Assays

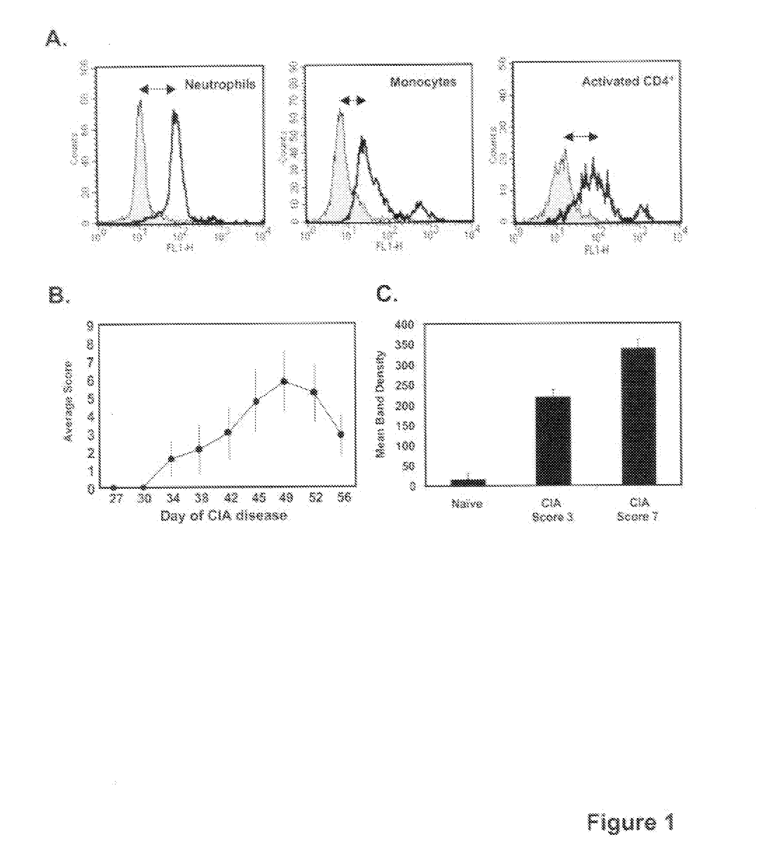

Macrophages were purified from mouse spleens using MACS CD11b+ positive selection. Purified macrophages (2×10⁴ per well) were co-cultured with MC57 fibroblasts (104 per well) in RPMI+10% FBS in a 48-well plate with a total volume of 500 μl/well. Following a 72-hour incubation at 37° C., levels of pro-MMP-9 in supernatants were measured with an ELISA kit (R&D systems). For blocking experiments, MC57 cells were incubated with 10 μg/ml anti-CD147 (either RL73.2 or UM-8D6), or their respective isotype control mAbs, for 15 minutes at room temperature before adding purified macrophages. Referring now to the figures, FIG. 1. CD147 expression and CypA in CIA. (A) Neutrophils, monocytes and activated CD4+ T cells were stained with anti-CD147, or isotype control antibody, followed by FITC-conjugated anti-rat IgG. Neutrophils and monocytes were distinguished based on forward scatter/side scatter characteristics and CD4+ T cells were identified by co-staining with Cy5-anti-CD4. Histograms show CD147 staining (open) versus isotype staining (filled). (B) DBA/1J mice were immunized with collagen in CFA and then challenged three weeks later with collagen in PBS. Starting 7 days after challenge, inflammation was scored for individual joints. Data show mean (+SE) clinical scores with n=6 mice per time point. (C) Groups of mice were sacrificed when they reached specific CIA scores and proteins extracted from joints were analyzed by Western blot analysis using anti-CypA Ab. Data showed mean band densitometry of lysates from naïve mice vs. mice with intermediate or high CIA clinical scores.

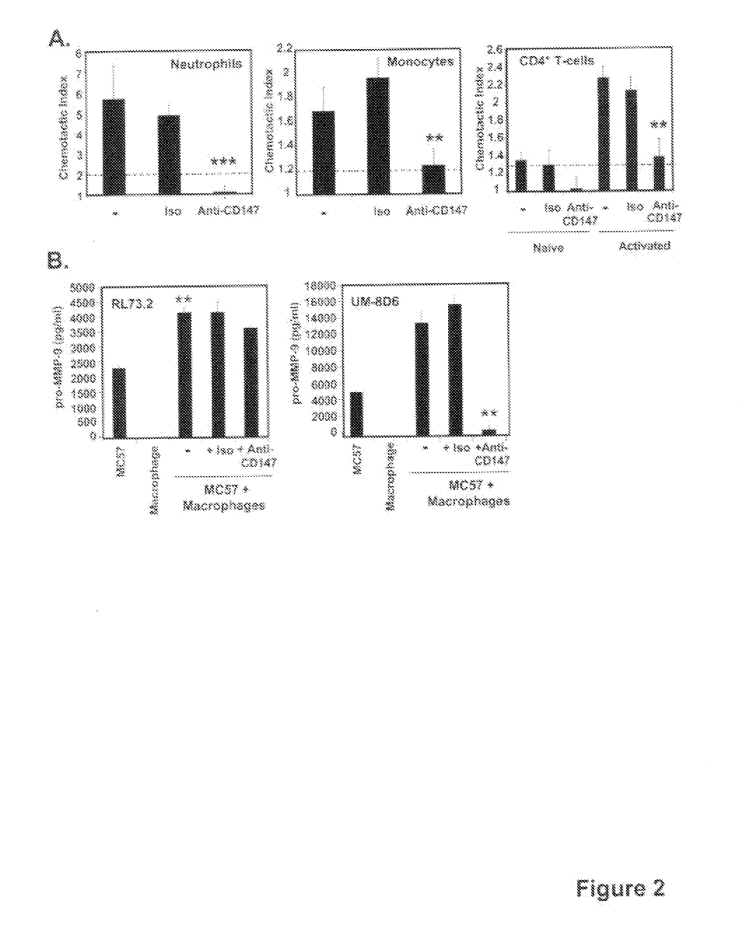

FIG. 2 shows that Leukocyte migration mediated by CypA was blocked by RL73.2 anti-CD147. (A) Purified neutrophils, monocytes and CD4+ T cells were set up in Boyden chemotaxis chambers in the presence of extracellular CypA with/without anti-CD147 or isotype control mAb. Bar graphs show mean (+SE) chemotactic index for each group, with n=4-6 wells per group. Horizontal dashed lines denote the cutoff chemotactic index for significant migration, set at 1.25. (B) MC57 mouse fibroblasts and purified mouse macrophages were co-cultured in the presence of RL73.2 anti-CD147, or UM-8D6 anti-CD147, or relevant isotype control mAbs. After 72 hours, culture supernatants were tested for pro-MMP-9 by ELISA. Bar graphs show mean (+SE) concentrations of pro-MMP-9 detected in cultures. Statistical differences between anti-CD147 and isotype groups were established using a Student's t-test: ***=p<0.001 and **=p<0.01.

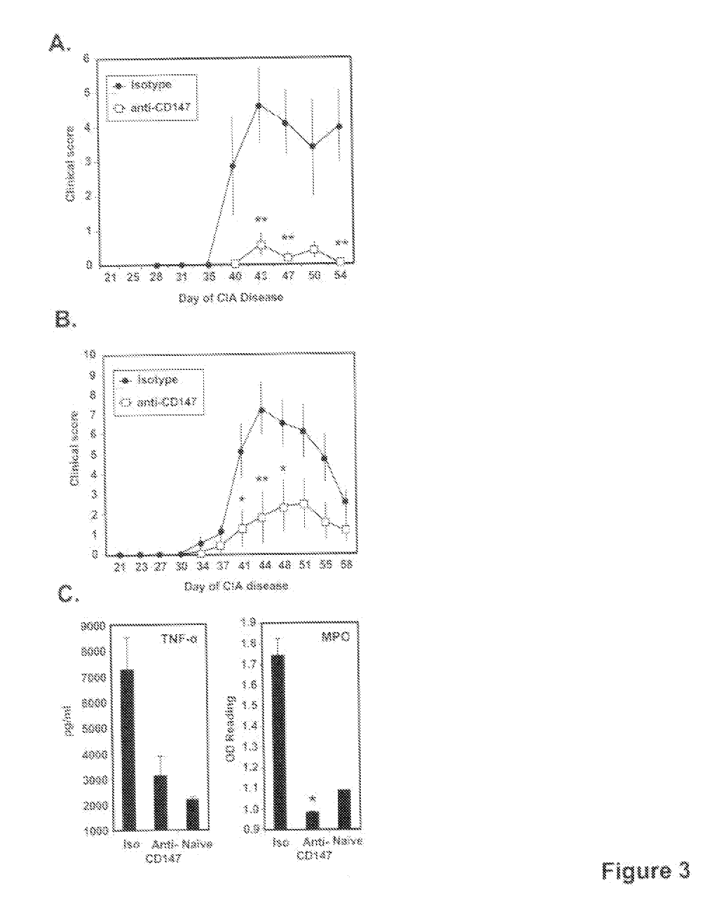

FIG. 3 Anti-CD147 intervention reduces the severity of CIA. DBA1/J mice were immunized and challenged with collagen II to induce CIA. (A) Half the mice were treated every 3 days starting on day 21 with RL73.2 anti-CD147 mAb while the other half was treated with isotype control mAb. Graph shows the mean (+SE) score for each group (n=6 per group). (B) Half the mice were treated on days 21-30 with RL73.2 anti-CD147 mAb while the other half were treated with an isotype control mAb. Graph shows the mean (+SE) score for each group (n=9 per group). (C) Mice were sacrificed at the peak differential between anti-CD147 and isotype treatment and joint proteins were extracted. MPO levels were established using TMB substrate and TNFα levels were measured by ELISA. A Student's t-test was used to establish statistical significance at individual time points or between groups: **=p<0.01; *=p<0.05.

### Example 9

### Non-Immunosuppressive Cyclosporine Analogs

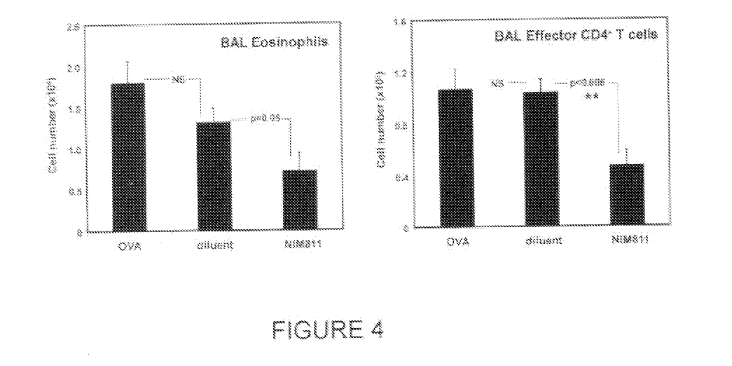

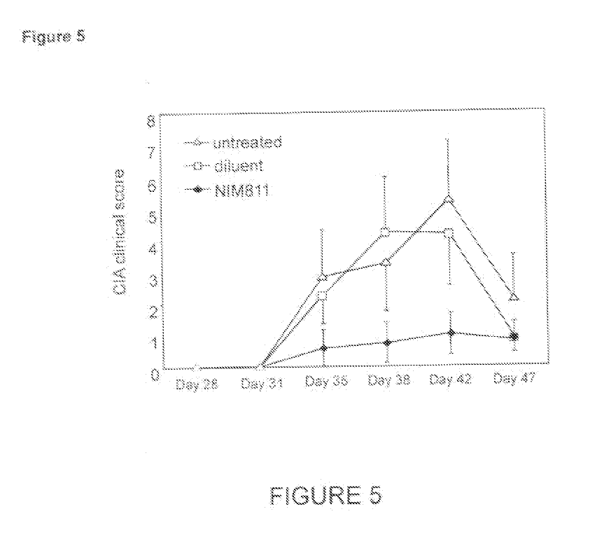

Intervention in Mouse Model of Allergic Asthma using non-immunosupressive CsA. Referring to FIG. 4, a non-immunosuppressive analog of CsA (NI-CsA) is effective at interfering with CD147-cyclosporin interactions. Mice were primed with OVA/Alum and challenged with intranasal OVA on days 7-10. Some mice were also given NI-CsA, or diluent alone, days 7, 9 and 11. On day 12 all animals were sacrificed and BAL leukocytes counted and stained for eosinophils (CCR3) and effector/memory T cells (CD4/CD62L). Data show cell numbers for each group (n=5-6), with t-test analysis used to establish statistical significance. [0083] EXAMPLE—NI-CsA Intervention in Mouse Model of CIA—Early Treatment [0084] Referring now to FIG. 5, the NI-CsA is again shown effective at interfering with CD147-cyclophilin interactions. NI-CsA treatment reduces joint inflammation: Male DBA/1J mice were immunized with collagen II in CFA at the base of the tail and 3 weeks later the mice were boosted i.p. with collagen II in PBS. Groups of mice then received either NI-CsA (200 μg per dose), diluent alone, or nothing, every two days from day 21-31. The diluent used was 15% Cremophor EL. Graph shows the mean±SE CIA clinical score for each group (n=6-8 mice per group) at different days post initial immunization.

### Example 9

### NI-CsA Intervention in Mouse Model of CIA—Later Treatment

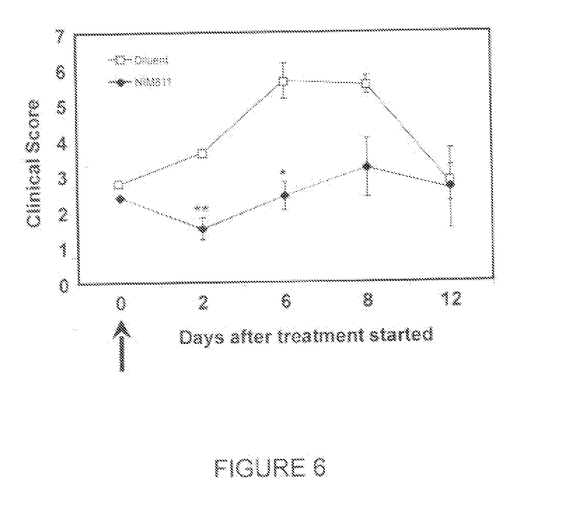

Referring to FIG. 6, NI-CsA is again shown effective at interfering with CD147-cyclophilin interactions. NI-CsA treatment reduces ongoing joint inflammation: male DBA/1J mice were immunized with collagen II in CFA at the base of the tail and 3 weeks later the mice were boosted i.p. with collagen II in PBS. Groups of mice then received either NI-CsA (200 μg per dose) or diluent alone every two days starting from the day they reached a CIA clinical score of 2. Graph shows the mean±SE CIA clinical score for each group (n=4-5 mice per group) starting from the day of treatment.

### Example 10

### Isolation of Human Leukocytes and Flow Cytometric Analysis

Blood was obtained from healthy human donors by venipuncture and the PBMCs enriched by centrifugation over Lymphocyte Separating Medium (LSM; Mediatech Inc., Herndon, Va.). Isolation of CD4+ T cells was conducted by MACS separation using a negative depletion kit (Miltenyi Biotec, Aurora, Calif.). Neutrophils were enriched from blood by centrifugation over Histopaque 1077 (Sigma Aldrich, St. Louis, Mo.), followed by differential sedimentation in 3% dextran solution. For isolation of PBLs, red blood cells were removed by water lysis of whole blood.

For flow cytometric analysis, PBMCs or PBLs were co-stained with TriColor-conjugated anti-CD4 or APC-conjugated anti-CD14 and FITC-anti-human CD147 (Research Diagnostics, Flanders, N.J.) or FITC-IgG1 isotype control mAb. For studies looking at activation markers, PE-labeled anti-CD25 or PE-labeled anti-HLA-DR were included. Staining was conducted on ice for 30 minutes followed by fixation in 1% paraformaldehyde. Flow cytometric analysis was done using a FACSCalibur instrument and CELLQuest software (Becton Dickinson, San Jose, Calif.).

### Example 11

### In vitro Activation of T Lymphocytes

PBMCs were suspended in tissue culture medium (Clicks medium containing 5% FCS) at 3×10⁶ per ml in the presence of 1 μg/ml PHA (Sigma-Aldrich) or 1 ng/ml SEA (Sigma-Aldrich). The cultures were incubated at 37° C. for 24-48 hours. Cells recovered from PHA or SEA cultures were centrifuged over LSM to remove dead cells and debris and then either used immediately for flow cytometric staining or were enriched for CD4+ T cells by MACS separation for chemotaxis assays or Western blot analysis. For studies comparing activated versus non-activated CD4+ T cells, a fresh blood draw from the same donor was used as the source of non-activated cells on the day of the assay. For studies in which heparan sulphate receptors were removed, populations of PHA-activated and non-activated PBMC were treated for 3 hours at 37° C. with heparinase I (Sigma-Aldrich) at 2 units per 10⁶ cells, prior to CD4+ T cell purification.

### Example 12

### CD147 is Upregulated on Activated Human CD4+ T Cells

In initial studies we established the baseline expression of CD147 on circulating non-activated versus activated CD4+ T cells. For these studies, human PBMC were co-stained with anti-CD4 and anti-CD25 or anti-HLA-DR (markers associated with the activation status of lymphocytes) plus anti-human CD147 or isotype control mAb. We have found that while CD147 was expressed at a high level on all CD4+ T cells, CD25+ and HLA-DR+ cells expressed the highest levels of CD147.

Among four different donors, the average CD147 mean fluorescence intensity (MFI) of CD4+ T cells with an activated status was 82.9+1.8 MFI units, compared to resting cells which was 53.6+1.2 units. This difference was highly significant (p<0.0001). The observation that activated CD4+ T cells expressed higher levels of CD147 than resting cells was confirmed by generating populations of CD4+ T cells enriched for activated cells by in vitro activation using PHA. We also studied the expression of CD147 versus CD25 or HLA-DR on CD4+ T cells after 48 hours of PHA activation. As observed with peripheral CD4+ T cells, the CD147 bright subset of T cells co-segregated with elevated expression of CD25 and HLA-DR, confirming that activated CD4+ T cells express higher levels of CD147 than non-activated cells.

### Example 13

### Activated T Cells Show Enhanced Migration to Extracellular Cyclophilins

In order to address the correlation between CD147 expression and cyclophilin-mediated responses, we compared the capacity of recombinant CypA to induce migration in populations of resting CD4+ T cells versus populations with a high frequency of activated CD4+ T cells. For these studies, human PBMC were stimulated in vitro with either PHA or SEA to generate populations containing a high frequency of activated T cells. After activation, the CD4+ T cells were purified and compared with CD4+ T cells obtained from a fresh blood draw from the same donor, consisting of mostly (>90%) resting cells. The two pools of CD4+ T cells were set up in Boyden chambers in the presence of previously optimized dose of recombinant CypA, or medium alone. The activated population responded with a significantly greater chemotactic index than resting cells. These findings suggested that activated T cells more readily migrate in response to extracellular CypA than their resting counterparts and suggest that their interaction with CypA may be more sensitive. The elevated level of CD147 expression on activated CD4+ T cells was comparable to that on neutrophils and monocytes, suggesting a likely association between potent CypA-induced responses and elevated CD147 expression.

### Example 14

### Cyclophilin-Mediated Migration of Resting and Activated T Cells is CD147-Dependent

To address the importance of CD147 expression on the observed migration of resting and activated T cells to CypA, we conducted experiments in which anti-CD147 mAb (or IgG1 isotype mAb) was included in the chemotaxis assay. The presence of anti-CD147 mAb inhibited the CypA-mediated chemotactic responses of resting CD4+ T cells, as well as activated CD4+ T cells, by >90%. Importantly, anti-CD147 mAb had no impact on the chemotactic responses of the same cells to RANTES, confirming the specificity of cyclophilin-CD147 interactions in activated CD4+ T cells. Further evidence that these interactions might be more potent in activated, compared to resting, CD4+ T cells was obtained in experiments looking at the requirement for co-interaction with cell surface heparan sulphate receptors. Extracellular cyclophilins must initially bind to cell surface heparan sulphate proteoglycans before they can interact with CD147 signaling receptors. A pre-incubation of human neutrophils or resting T lymphocytes with heparinase was shown to inhibit cyclophilin-mediated signaling events by >90%. Strikingly, in our current studies we observed that, while heparinase treatment of non-activated (resting) CD4+ T cells resulted in a >95% inhibition in CypA-induced migration, the same treatment had no impact on the migration of activated CD4+ T cells. Primary T cells, including activated CD4+ T cells, have been shown to express very low levels of heparans, relative to other leukocyte subsets, so it unlikely that the heparinase treatment was ineffective at removing all cell surface heparan sulphates from the activated T cells. Indeed, the same heparinase regimen abrogated the capacity of neutrophils, known to require heparans co-interaction to respond to cyclophilin A. Furthermore, activated CD4+ T cells treated with a five-fold greater dose of heparinase still demonstrated a potent migration to cyclophilin A.

To establish whether the cyclophilin-induced migration of activated CD4+ T cells treated with heparinase was still dependent on interaction with CD147 receptors, chemotaxis assays were conducted with heparinase-treated CD4+ T cells in the presence of anti-CD147 mAb. The results suggested that anti-CD147 antibody inhibited the migration of heparinase-treated activated CD4+ T cells by >90%, demonstrating a continued dependence on CD147 for migration to extracellular cyclophilins. Taken together, these findings suggest that the interactions that regulate cyclophilin-mediated migration in T cells may be less stringent in activated, compared to resting, cells and that CD147 alone may be sufficient to induce both the binding and signaling events required for activated T cell migration/recruitment to extracellular cyclophilins.

### Example 15

### CD147 Expression and CypA in CIA

In preparation for our in vivo studies investigating how CD147-extracellular cyclophilin interactions contribute to collagen induced arthritis (CIA), we first established that the two components of the interaction, CD147 and cyclophilins, were present in mouse CIA. The expression profile of CD147 on three subsets of pro-inflammatory leukocytes is known to contribute to tissue pathology during CIA and RA, specifically neutrophils, monocytes, and activated CD4+ T cells. Flow cytometric analysis confirmed that CD147 was readily detectable on all three subsets. We next confirmed that elevated levels of cyclophilins were present in the inflamed joints of mice with CIA, as observed in the joints of human RA patients. We studied the time course of joint inflammation induced during CIA, based on a macroscopic scoring system. Since inflammation in mouse CIA is restricted to wrist and ankle joints where the synovial space cannot be sampled directly, changes in cytokines and chemokines are typically examined in homogenized joint tissue.

Thus, proteins were extracted from the joints of CIA mice sacrificed at various clinical scores and Western blot analysis was performed to establish the presence of CypA. We detected that levels of CypA were elevated in the joints of mice with CIA relative to naive control mice, and the levels increased with increasing clinical scores. We acknowledge that it is not possible for us to conclude that the observed increases in CypA were solely due to changes in extracellular CypA, since the proteins analyzed are a mixture of intracellular, as well as extracellular CypA. However, using RT-PCR for CypA mRNA, we established that the differences in levels of CypA proteins induced during CIA are not the result of a per cell increase in mRNA transcription. Therefore, the increase in synovial CypA protein observed during CIA is likely due to an increase in numbers of cells expressing and/or secreting CypA. Leukocyte migration mediated by CypA is blocked by RL73.2 CD147 antibody.

In order to establish the contribution of CD147-cyclophilin interactions in the context of leukocyte recruitment, we next confirmed that treatment with CD147 mAb could directly inhibit the migration induced by CypA of CIA-relevant leukocytes. For these studies, we made use of the RL73.2 clone of anti-mouse CD147. We have used this clone successfully to reduce the influx of leukocytes during acute lung inflammation in vivo. We have shown that neutrophils, monocytes, and activated CD4+ T-cells all migrated well to CypA. Interestingly, naïve CD4+ T cells, a subset of leukocytes known to be poorly recruited into tissues, displayed minimal migration.

We have previously shown that both human and mouse naïve CD4+ T cells express significantly lower levels of cell surface CD147 than activated CD4+ T cells, providing an explanation for their poor capacity to interact with, and respond to, CypA. In the case of neutrophils, monocytes and activated CD4+ T cells, the observed migration induced by CypA was inhibited by >90% by the RL73.2 mAb. Taken together, these findings demonstrated the capacity of the RL73.2 anti-CD147 clone to directly interfere with the CypA-mediated recruitment of pro-inflammatory leukocytes.

RL73.2 antibody does not impact on the EMMPRIN function of CD147. One of the best-established functions of CD147 is its role as an inducer of matrix metalloproteinases. Given the importance of MMPs in tissue destruction and remodeling during RA, we examined whether the RL73.2 antibody used to inhibit the chemotactic function of CD147 might also impact on its EMMPRIN function. The co-culture of mouse MC57 fibroblasts with primary mouse macrophages induced an increase in the secretion of pro-MMP-9, the precursor for active MMP-9. However, the presence of CD147 mAb RL73.2 during culture had no impact on the augmented secretion of pro-MMP-9, regardless of the dose tested. This was in marked contrast to a different anti-CD147 clone, UM-8D6, that reduced pro-MMP-9 secretion by >90%. These findings suggest that the two clones interact with different functional domains of CD147. The extracellular portion of CD147 consists of two immunoglobulin domains (domains 1 and 2) that are thought to mediate different functions. Thus, the two different clones are likely binding to, and interfering with, one or the other of these two domains. From the current findings, we conclude that RL73.2 anti-CD147 mAb has the capacity to inhibit the chemotactic function of CD147, but not its EMMPRIN function.

### Example 16

### Treatment with RL73.2 Antibody Significantly Reduces CIA Severity

We examined the impact of targeting the chemotactic function of CD147 during CIA. In these studies, RL73.2 mAb was administered to CIA mice throughout the course of disease, starting from the day of collagen challenge and then every 3 days until sacrifice. Strikingly, anti-CD147 treatment almost completely prevented joint inflammation from being established. While inflammation was noted at some time points (days 43 and 50), the severity of this inflammation was >90% lower that that in isotype-treated control mice. The half-life of rat IgG2a antibodies (the isotype of RL73.2) is estimated to be >105 hours.

Further, it was investigated whether administering anti-CD147 only at the onset of disease, rather than throughout, would be sufficient to cause a significant reduction in joint inflammation. For these studies CIA mice were given RL73.2 mAb for 10 days, starting from the time of challenge, and then treatment was stopped. As shown in FIG. 3B, these anti-CD147 treated mice demonstrated delayed CIA-mediated joint inflammation, as well as a highly significant reduction (>75%) in clinical scores, compared to mice treated with an isotype control antibody. In a repeat of this shortened intervention regimen, some mice were sacrificed at the peak differential between anti-CD147 and isotype clinical scores (in this experiment: day 42, showing 80% reduction in inflammation) and joints were collected for protein extraction. These were then assayed for the presence of myeloperoxidase (MPO) and TNF-α, products that are secreted by infiltrating neutrophils and/or macrophages. Results demonstrated that both products were markedly reduced in the joints of mice receiving RL73.2 intervention.

The pathology of rheumatoid arthritis is characterized by two distinct phases. During the initiation phase, antigen-specific T and B cells are activated and expand within the synovium. The subsequent effector phase is defined by the recruitment of neutrophils, monocytes, and lymphocytes into the joint where, in conjunction with resident fibroblasts, they contribute to the destructive events of the disease through the production of proteases, cytokines, and other mediators. Our findings of a reduction in factors associated with the presence of activated neutrophils suggested that anti-CD147 intervention is likely impacting on the effector phase of disease. This conclusion is further supported by our findings that providing the intervention regimen throughout the course of disease almost completely suppressed the development of joint inflammation. These observations fit well with the fact that significant increases in CypA within joints are only observed once the inflammatory response is underway, arguing that any cyclophilin-mediated recruitment of leukocytes would be induced during the effector phase, rather than during the initiation phase, of the response.

Accordingly, these studies have provided evidence that targeting the chemotactic function of CD147 can have a significant impact on the development and severity of joint inflammation in CIA. Although other types of intervention have also proven successful in reducing tissue inflammation in CIA/RA, many of these target immune components that under normal circumstances have a beneficial immunological role to play. For example, while anti-TNFα may be highly effective at reducing joint inflammation in RA, the lack of this cytokine during infection can be detrimental to the host. Indeed, the delivery of anti-TNFα monoclonal antibodies to humans has been reported to increase the risk of serious infection and the development of malignancies. Thus, it is contemplated herein that targeting CD147 and its interaction with cyclophilins may provide a less detrimental therapeutic approach because elevated levels of extracellular cyclophilins are only present during situations of prolonged/persistent tissue inflammation. Thus, blocking the capacity of CD147 to interact with extracellular cyclophilins will impact only on the leukocyte recruitment that is mediated by long-lived inflammation, such as during chronic inflammatory diseases.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable equivalents.

### REFERENCES

1. Bukrinsky, M. I. (2002) Cyclophilins: unexpected messengers in intercellular communications. Trends Immunol. 23, 323-325.
2. Yurchenko, V., Constant, S., Bukrinsky, M. (2005) Dealing with the family: CD147 interactions with cyclophilins. Immunology 117, 301-309.
3. Yurchenko, V., Zybarth, G., O'Connor, M., Dai, W. W., Franchin, G., Hao, T., Guo, H., Hung, H. C., Toole, B., Gallay, P., Sherry, B., Bukrinsky, M. (2002) Active-site residues of cyclophilin A are crucial for its signaling activity via CD147. J. Biol. Chem. 277, 22959-22965.
4. Sherry, B., Yartlett, N., Strupp, A., Cerami, A. (1992) Identification of cyclophilin as a proinflammatory secretory product of lipopolysaccharide-activated macrophages. Proc. Natl. Acad. Sci. USA 89, 3511-3515.
5. Xu, Q., Leiva, M. C., Fischkoff, S. A., Handschumacher, R. E., Lyttle, C. R. (1992) Leukocyte chemotactic activity of cyclophilin. J. Biol. Chem. 267, 11968-11971.
6. Billich, A., Winkler, G., Aschauer, H., Rot, A., Peichl, P. (1997) Presence of cyclophilin A in synovial fluids of patients with rheumatoid arthritis. J. Exp. Med. 185, 975-980.
7. De Ceuninck, F., Allain, F., Caliez, A., Spik, G., Vanhoutte, P. (2003) High binding capacity of cyclophilin B to chondrocyte heparan sulfate proteoglycans and its release from the cell surface by matrix metalloproteinases: possible role as a proinflammatory mediator in arthritis. Arthritis Rheum. 48, 2197-2206.
8. Jin, Z. G., Melaragno, M. G., Liao, D. F., Yan, C., Haendeler, J., Suh, Y. A., Lambeth, J. D., Berk, B. C. (2000) Cyclophilin A is a secreted growth factor induced by oxidative stress. Circ. Res. 87, 789-796.
9. Tegeder, I., Schumacher, A., John, S., Geiger, H., Geisslinger, G., Bankg, H., Brune, K. (1997) Elevated serum cyclophilin levels in patients with severe sepsis. J. Clin. Immunol. 17, 380-386.
10. Konttinen, Y. T., Li, T. F., Mandelin, J., Liljestrom, M., Sorsa, T., Santavirta, S., Virtanen, I. (2000) Increased expression of extracellular matrix metalloproteinase inducer in rheumatoid synovium. Arthritis Rheum. 43, 275-280.
11. Zhu, P., Ding, J., Zhou, J., Dong, W.-J., Fan, C.-M., Chen, Z.-N. (2005) Expression of CD147 on monocytes/macrophages in rheumatoid arthritis: its potential role in monocyte accumulation and matrix metalloproteinase production. Arthritis Res. & Therapy 7, R1023-R1033.
12. Zhu, P., Lu, N., Shi, Z.-G., Zhou, J., Wu, Z.-B., Yang, Y., Ding, J., Chen, Z. (2006) CD147 overexpression on synoviocytes in rheumatoid arthritis enhances matrix metalloproteinase production and invasiveness of synoviocytes. Arthritis Res. & Therapy 8, R44.
13. Arora, K., Gwinn, W., Bower, M., Watson, A., Okwumabua, I., MacDonald, H., Bukrinsky, M., Constant, S. (2005) Extracellular cyclophilins contribute to the regulation of inflammatory responses. J. Immunol. 175, 517-522.
14. Gwinn, W., Damsker, J., Falahati, R., Okwumabua, I., Kelly-Welch, A., Keegan, A., Vanpouille, C., Lee, J., Dent, L. A., Leitenberg, D., Bukrinsky, M., Constant, S. (2006) Novel approach to inhibit asthma-mediated lung inflammation using anti-CD147 intervention. J. Immunol. 177, 4870-4879.
15. Allain, F., Vanpouille, C., Carpentier, M., Slomianny, M. C., Durieux, S., Spik, G. (2002) Interaction with glycosaminoglycans is required for cyclophilin B to trigger integrin-mediated adhesion of peripheral blood T lymphocytes to extracellular matrix. Proc. Natl. Acad. Sci. USA 99, 2714-2719.
16. Mackay, C. R. (2000) Follicular homing T helper (Th) cells and the Th1/Th2 paradigm. J. Exp. Med. 192, F31-F34.
17. Salmon, M., Hill-Gaston, J. (1995) The role of T-lymphocytes in rheumatoid arthritis. British Med. Bulletin 51, 332-345.
18. Grabie, N., Delfs, M., Westrich, J., Love, V., Stavrakis, G., Ahmad, F., Seidman, C., Seidman, J., Lichtman, A. (2003) IL-12 is required for differentiation of pathogenic CD8⁺ T cell effectors that cause myocarditis. J. Clin. Invest. 111, 671-680.
19. Saphire, A., Bobardt, M., Zhang, Z., David, G., Gallay, P. (2001) Syndecans serve as attachment receptors for human immunodeficiency virus Type 1 on macrophages. J. Virol. 75, 9187-9200.
20. Vanpouille, C., Denys, A., Carpentier, M., Pakula, R., Mazurier, J., Allain, F. (2004) Octasaccharide is the minimal length unit required for efficient binding of cyclophilin B to heparin and cell surface heparan sulphate. Biochem. J. 382, 733-740.
21. Tomita, T., Nakase, T., Kaneko, M., Shi, K., Takahi, K., Ochi, T., Yoshikawa, H. (2002) Expression of extracellular matrix metalloproteinase inducer and enhancement of the production of matrix metalloproteinases in rheumatoid arthritis. Arthritis Rheum. 46, 373-378.
22. Kasinrerk, W., Fiebiger, E., Stefanova, I., Baumruker, T., Knapp, W., Stockinger, H. (1992) Human leukocyte activation antigen M6, a member of the Ig superfamily, is the species homologue of rat OX-47, mouse basigin, and chicken HT7 molecule. J. Immunol. 149, 847-854.
23. Koch, C., Staffler, G., Huttinger, R., Hilgert, I., Prager, E., Cemy, J., Steinlein, P., Majdic, O., Horejsi, V., Sockinger, H. (1999) T cell activation-associated epitopes of CD147 in regulation of the T cell response, and their definition by antibody affinity and antigen density. Int. Immunol. 11, 777-786.

We claim:

1. A method for treatment or amelioration of one or more symptoms of a chronic inflammatory disease in an individual in need thereof comprising, administering a therapeutically effective amount of a compound that blocks CD147 interaction with an extracellular cyclophilin without blocking the CD147 domain involved with EMMPRIN function, wherein the compound regulates recruitment of leukocyte to a target tissue and thereby treat or ameliorate symptoms of the chronic inflammatory disease.

2. The method of claim 1, wherein the compound is a derivative of the CD147, a derivative of the extracellular cyclophilin, or a combination thereof.

3. The method of claim 2, wherein the derivative of CD147 comprises an antagonist of the CD147.

4. The method of claim 3, wherein the antagonist of the CD147 comprises a monoclonal antibody against CD147.

5. The method of claim 1, wherein the cyclophilin comprises a derivative of cyclosporine A (CsA).

6. The method of claim 1, wherein the inflammatory disease comprises Osteoarthritis, Chronic Obstructive Pulmonary Disease, chronic inflammatory connective tissue diseases, lupus, scleroderma, Sjogrens' syndrome, poly- and dermatomyositis, vasculitis, chronic inflammatory bowel disease, multiple sclerosis, rosacea, chronic pelvic inflammatory disease, Crohn's disease, chronic inflammatory polyneuropathy, Rheumatoid arthritis, and neovascular diseases of the eye, among others.

7. The method of claim 1, wherein the compound inhibits or reduces recruitment of leukocyte to a target tissue.

8. The method of claim 1, wherein the compound is a non-immunosuppressive derivative of cyclosporine A modified to inhibit interactions of intracellular cyclophilin.

9. A drug screening method for selecting compounds that inhibit binding between CD147 and external cyclophilins without blocking the CD147 domain involved with EMMPRIN comprising, contacting the CD147 or cyclophilin polypeptide or a variant thereof with a library of compounds suspected of having antagonist or agonist activity with CD147 and/or cyclophilins, identifying select compounds that binds to otherwise interacts CD147 and/or cyclophilins, and assaying the biological activity of the select compounds, wherein the selected compound reduces or inhibits recruitment of leukocyte to a target tissue.

10. A pharmaceutical composition for treatment of a chronic inflammatory disease in an individual in need thereof comprising, a therapeutically effective amount of a compound that blocks binding between CD147 and an extracellular cyclophilin without blocking the CD147 domain involved with EMMPRIN function, and a suitable carrier or diluent.

11. The pharmaceutical composition of claim 10, wherein the compound is a derivative of the CD147, a derivative of the extracellular cyclophilins, or a combination thereof.

12. The pharmaceutical composition of claim 10, wherein the derivative of CD147 comprises an antagonist of the CD147.

13. The pharmaceutical composition of claim 10, wherein the antagonist of the CD147 comprises a monoclonal antibody against CD147.

14. The pharmaceutical composition of claim 10, wherein the inhibitor comprises a derivative of cyclosporine A (CsA).

15. The pharmaceutical composition of claim 10, wherein the inflammatory disease comprises Osteoarthritis, Chronic Obstructive Pulmonary Disease, chronic inflammatory connective tissue diseases, lupus, scleroderma, Sjogrens' syndrome, poly- and dermatomyositis, vasculitis, chronic inflammatory bowel disease, multiple sclerosis, rosacea, chronic pelvic inflammatory disease, Crohn's disease, chronic inflammatory polyneuropathy, Rheumatoid arthritis, and neovascular diseases of the eye, among others.

16. The pharmaceutical composition of claim 10, wherein the composition inhibits or reduces recruitment of leukocyte to a target tissue.

17. The pharmaceutical composition of claim 10, wherein the compound is a non-immunosuppressive derivative of cyclosporine A (CsA) modified to inhibit its interaction with intracellular cyclophilin.

18. A diagnostic test kit for to diagnose, detect or quantify an inflammatory disease in a subject in need thereof, comprising a compound that blocks binding between CD147 and an extracellular cyclophilin without blocking the CD147 domain involved with EMMPRIN function, inactive agents used for biological assays including buffers, minerals, and water, among others, instructions for the use of the compound and the biological assay to predict or diagnose the occurrence or recurrence of an inflammatory mediated disease in the subject.

19. The diagnostic test kit of claim 18, wherein the compound is a derivative of the CD147, a derivative of the extracellular cyclophilin, or a combination thereof.

20. The diagnostic test kit of claim 18, wherein the inhibitor of CD147 comprises an antagonist of the CD147.
